(19) United States
(12) Patent Application Publication
(10) Pub. No.: US 2006/0286180 A1
(43) Pub. Date: Dec. 21, 2006

(54) Lipolysis promoter and food and drink containing the same

(76) Inventors: Yoko Suetake, Saitama (JP); Keishiro Yoshida, Saitama (JP); Susumu Shimura, Saitama (JP); Tsutomu Arakawa, Saitama (JP); Masahiro Tani, Saitama (JP)

Correspondence Address: YOUNG & THOMPSON, 745 SOUTH 23RD STREET, 2ND FLOOR, ARLINGTON, VA, 22202, US

(21) Appl. No.: 11/451,399

(22) Filed: Jun. 13, 2006

(30) Foreign Application Priority Data
Jun. 16, 2005 (JP) 2005-177058

(51) Int. Cl. A61K 36/71; A61K 36/896; A61K 36/66; A61K 36/28

(52) U.S. Cl. 424/725; 424/726; 424/749; 424/753; 424/764

(57) ABSTRACT

[Problem to be solved] To provide a naturally-derived lipolysis promoter with high safety, which may promote the degradation of the accumulated adipose tissue to control, prevent, and ameliorate obesity to a satisfactory extent.

[Solution] A lipolysis promoter containing one kind, or two or more kinds of plant bodies and/or extracts therefrom selected from the group consisting of Myristica fragrance, Symplocarpus foetidus, Escholzia california, Sparganium stoloniferum, Sanguinaria canadensis, Mahonia Aquifolium, Acorus gramineus, the fruit site of Musa paradiciaca, Cytisus scoparius, Hydrastis canadensis, Ficaria ranunculoides, Fumaria officinalis, Unonopsis floribunda, Nasturtium officinale, Nigella sativa, Urtica dioica, Capsella bursa-pastoris, and Polygonum bistorta as active ingredients.

### TECHNICAL FIELD TO WHICH THE INVENTION PERTAINS

The present invention relates to a lipolysis promoter which promotes the degradation of body fat, reduces systemic or local fat, and is effective in preventing and ameliorating obesity, and food and drink containing the same.

### BACKGROUND ART

Obesity results from the accumulation of intake energy in adipocytes as neutral fat in excess of consumption energy, and not only triggers various diseases such as arteriosclerosis, but also is cosmetically undesirable, so that its prevention and amelioration are strongly required. However, recent years have seen an increase in the obesity rate in the industrialized countries including our country year by year. The reason comes from overeating, lack of exercise, stress, and the like, but there are many people who cannot continue dietary restriction and exercise therapy, which require a strong will and long continuance.

This background has given rise to a wide range of development of lipolysis promoters effective in preventing and ameliorating the obesity. For example, it is known that a lipolysis promoter with an extract from at least one or more kinds of raw materials selected from the group consisting of Coix lachryma-jobi L. var. ma-yuen Stapf, Hordeum vulgare, Cassia obtusifolia, Psidium guajava Linne, and Camellia sinensis as an active ingredient promotes the degradation of fat accumulated in the adipocytes to contribute to control and prevention of the obesity (for example, refer to Patent Document 1). It is also known that a lipolysis promoter containing one kind, or two or more kinds selected from the group consisting of the extract of Geranium nepalense subsp. thunbergii, the extract of Paeonia lactiflora Pall, the extract of Swertia japonica, and the extract of Thymus vulgaris is effective in reforming obese constitution by promoting reduction in systemic or local adipose tissue, or in controlling or preventing the obesity by preventing the above tissue growth (for example, refer to Patent Document 2). It is further known that a lipolysis promoter formed containing at least one kind selected from the group consisting of Citrus aurantium, Citrus sinensis, Citrus vulgaris, Tussilago farfara, and Triticum vulgare as an active ingredient is effective in controlling or preventing the obesity, reforming the obese constitution, and reducing the systemic or local adipose tissue (for example, refer to Patent Document 3). It is furthermore known that a lipolysis promoter characterized by containing a piperaceous plant as an active ingredient has apparent lipolysis promoting activity in the adipose tissue, and a beneficial effect on controlling, preventing and ameliorating the obesity (for example, refer to Patent Document 4). It is still further known that a lipolysis promoter characterized by containing a Cirsium plant as an active ingredient has the apparent lipolysis promoting activity in the adipose tissue, and a beneficial effect on controlling, preventing, and ameliorating the obesity (for example, refer to Patent Document 5). It is yet further known that a lipolysis promoter containing banana pericarp or its extract may promote the degradation of the accumulated fat to control, prevent, and ameliorate the obesity to a satisfactory extent (for example, refer to Patent Document 6). However, these lipolysis promoters do not always have a satisfying effect, and some of them are concerned to produce side effects.

[Patent Document 1] Japanese Laid-open Patent Publication No. 2002-275078

[Patent Document 2] Japanese Laid-open Patent Publication No. 2000-63237

[Patent Document 3] Japanese Laid-open Patent Publication No. 11(1999)-228431

[Patent Document 4] Japanese Laid-open Patent Publication No. 8(1996)-245410

[Patent Document 5] Japanese Laid-open Patent Publication No. 8(1996)-301780

[Patent Document 6] Japanese Laid-open Patent Publication No. 2000-44482

### DISCLOSURE OF THE INVENTION

### Problems to be Solved by the Invention

With the foregoing background, there is required further development of a lipolysis promoter having a satisfying effect on preventing and ameliorating obesity, and capable of safe usage.

### MEANS FOR SOLVING THE PROBLEMS

The present inventor et al. have invented that Myristica fragrance, Symplocarpus foetidus, Escholzia california, Sparganium stoloniferum, Sanguinaria canadensis, Mahonia Aquifolium, Acorus gramineus, the fruit site of Musa paradiciaca, Cytisus scoparius, Hydrastis canadensis, Ficaria ranunculoides, Fumaria officinalis, Unonopsis floribunda, Nasturtium officinale, Nigella sativa, Urtica dioica, Capsella bursa-pastoris, and Polygonum bistorta have an effect on promoting the degradation of neutral lipid in adipocytes, and brought the present invention to completion as a result of taking note of increased and enlarged adipocytes, which trigger obesity, and making diligent studies of various plants based on an assumption that the obesity can be prevented and ameliorated by promoting the degradation of neutral fat in the adipocytes.

More specifically, the present invention is a lipolysis promoter containing one kind, or two or more kinds of plant bodies and/or extracts therefrom selected from the group consisting of Myristica fragrance, Symplocarpus foetidus, Escholzia california, Sparganium stoloniferum, Sanguinaria canadensis, Mahonia Aquifolium, Acorus gramineus, the fruit site of Musa paradiciaca, Cytisus scoparius, Hydrastis canadensis, Ficaria ranunculoides, Fumaria officinalis, Unonopsis floribunda, Nasturtium officinale, Nigella sativa, Urtica dioica, Capsella bursa-pastoris, and Polygonum bistorta as active ingredients. The present invention is also food and drink containing the lipolysis promoter described above.

### EFFECTS OF THE INVENTION

The lipolysis promoter and the food and drink containing the same according to the present invention have apparent lipolysis promoting activity in adipose tissue, and have a beneficial effect on preventing or ameliorating obesity, and reforming obese constitution.

### BEST MODE FOR CARRYING OUT THE INVENTION

Hereinafter, the present invention will be described in detail. While a description will be given of a lipolysis promoter, food and drink containing the same, and a process for producing the same, as well as its efficacy, and the like, it is to be understood that the present invention is not intended to be limited by these examples.

The lipolysis promoter of the present invention may directly contain each plant as an active ingredient, but may contain a dried product, and further a powder-processed dry product as active ingredients. In addition, it may contain an extract from each plant as an active ingredient. This plant extract may be water, various kinds of organic solvents or a liquid extract from various kinds of organic solvents containing water, but may be a substance in which this liquid extract is evaporated to dryness by a normal drying process (for example, drying under reduced pressure, freeze-drying, and the like) or concentrated by a concentrating process. The kinds of organic solvents include ethanol, methanol, acetone, ethyl acetate, and hexane, but are not particularly limited. Furthermore, this plant extract may be subjected to purification treatment such as deodorization and decolorization within the bounds of not affecting the effectiveness thereof, as necessary.

The lipolysis promoter of the present invention can be used in any form of an oral preparation, an external preparation, and the like. Accordingly, the lipolysis promoter of the present invention may be made into an pharmaceutical preparation adapted for ease of use as an internal medicine, for example, putting this plant extract into granules with the use of an excipient, and the like, as appropriate. Moreover, the lipolysis promoter may locally reduce fat in these sites by direct application to the face and the abdomen, and thus may be used as lotion, gel, skin lotion, an ointment, a paste, a cataplasm, a plaster, a stick agent, a sheet agent, a bath agent, a tablet for body cleaning, and the like.

The compounding amount of the lipolysis promoter of the present invention may be selected from a wide range though being dependent on an adding form and a dosage form. For example, in the case of the external preparation, it is preferable that the compounding amount thereof be not less than 0.005% by weight (hereinafter, expressed simply by %), particularly 0.01 to 30% by weight in a composition on a solvent extraction dried product basis. In the case of the oral preparation, it is also preferable that the compounding amount thereof be 0.01 to 10 g, particularly 0.05 to 3 g per day for adults on the solvent extraction dried product basis.

Said lipolysis promoter is compounded in the food and drink of the present invention, in which compoundable food and drink is not particularly limited, and may be compounded in various forms of confectionery such as chewing gum, candies, and chocolate, health food, drinks, health drinks, flavoring, bread, and noodles. The food and drink in accordance with the present invention may take the form of health food, functional food, or food for specified health use which is given an obesity protective effect and an obesity ameliorating effect. The food and drink in accordance with the present invention can also be used in general diet. And, intake of such compounded food and drink allows amelioration of obesity and improvement in lifestyle-related disease derived from the obesity. In this case, it is preferable that the compounding amount of the lipolysis promoter be not less than 0.0001% by weight, particularly 0.01 to 99% by weight in the food and drink on the solvent extraction dried-product basis.

Hereinafter, while the present invention will be described in more detail with test examples, it is to be understood that the scope of the present invention is not intended to be limited by these examples.

### TEST EXAMPLE 1

The present test was carried out to obtain a plant extract from a plant body.

1) Sample Under Test

Eighteen kinds of plants shown in Table 1 were used.

2) Test Method

The plants were dried, 50% ethanol or 100 ml of water was added to 10 g of a dried body thereof, extraction treatment was carried out under agitation at 70° C. for two hours, the resultant extract was filtered, and then subjected to vacuum concentration, followed by being freeze dried to provide the corresponding plant extract.

3) Test Result

Each extract yield is shown in [Table 1].

TABLE 1
Plant sample extraction rate

| | 50% ethanol | |
|---|---|---|
| Plant material name | extraction | Water extraction |
| Symplocarpus | 23% | 23% |
| foetidus | | |
| Cytisus scoparius | 16% | 19% |
| Sanguinaria | 28% | 28% |
| canadensis | | |
| Escholzia california | 6% | 20% |
| Mahonia Aquifolium | 8% | 8% |
| Myristica fragrance | 21% | 21% |
| Acorus gramineus | 6% | 15% |
| Musa paradiciaca | 35% | 39% |
| (fruit site) | | |
| Hydrastis canadensis | 22% | 16% |
| Sparganium | 14% | 16% |
| stoloniferum | | |
| Ficaria ranunculoides | 30% | 22% |
| Fumaria officinalis | 20% | 28% |
| Unonopsis floribunda | 6% | 5% |
| Nasturtium officinale | 20% | 19% |
| Nigella sativa | 21% | 12% |
| Urtica dioica | 11% | 18% |
| Capsella | 19% | 20% |
| bursa-pastoris | | |
| Polygonum bistorta | 26% | 29% |

### TEST EXAMPLE 2

The present test was carried out to examine the lipolysis promoting activity of the plant extract obtained in Text Example 1.

1) Sample Under Test

Freeze dried plant extracts of 18 kinds of 50% ethanol extracts and seven kinds of water extracts, which were prepared in Test Example 1, were used alone or in a combination of two kinds or more thereof.

2) Test Method

(I) Adipocyte Culture

MC3T3-G2/PA6 cells, mouse-derived preadipocytes, were seeded in a 24-well plate so as to achieve 5×10⁴ cells/well, and incubated in a 10% fetal bovine serum (FBS) adding α-MEM culture medium in the presence of 5% CO₂ at 37° C. Immediately before the plate becomes confluent, the culture medium was replaced by a 10% FBS α-MEM culture medium to which dexamethasone, 3-isobutyl-1-methylxanthine, and glucose were added to induce differentiation to adipocytes. The incubation was performed for eight to nine days after the induction, and the test was carried out after adipocyte maturation.

(II) Lipolysis Activity Measurement Method

After culture supernatant was discarded, and the well was cleaned with PBS (−), the freeze dried plant extracts and Dulbecco's Phoshate Buffered Saline containing 2% BSA and 4.5 g/L glucose were added, and incubated for one hour. It should be noted that the amount of the freeze dried plant extracts was adjusted so that the final concentration in this reaction system is 100 μg/ml in any case of using the freeze dried plant extracts alone or in the combination of two kinds or more thereof. After the incubation, the supernatant was sampled, and the release amount of glycerol, lipolytic product, was measured using triglyceride E-Test Wako. It should be noted that a lipolysis promoting rate is a relative value with a control value (in the case of not adding the freeze dried plant extracts) expressed by the following equation as 100%.
Lipolysis promoting rate (%)=[A/B]×100

A: amount of released glycerol when adding the extracts

B: amount of released glycerol when adding no extracts

3) Test Result

The lipolysis promoting activity was determined on the basis of the lipolysis promoting rate found from the measurements of the amount of released glycerol produced by lipolysis. As shown in [Table 2], [Table 3], and [Table 4], when the plant extracts under test were added alone or in the combination of two kinds or more thereof, the lipolysis was apparently promoted compared with the case of no addition.

TABLE 2
Lipolysis promoting rate of 50% ethanol extract

| Plant material name | % |
|---|---|
| Symplocarpus foetidus | 2300 |
| Cytisus scoparius | 1500 |
| Sanguinaria canadensis | 1500 |
| Escholzia california | 1500 |
| Mahonia Aquifolium | 1100 |
| Myristica fragrance | 1000 |
| Acorus gramineus | 1000 |
| Musa paradiciaca (fruit site) | 900 |
| Hydrastis canadensis | 720 |
| Sparganium stoloniferum | 540 |
| Ficaria ranunculoides | 500 |
| Fumaria officinalis | 490 |
| Unonopsis floribunda | 450 |
| Nasturtium officinale | 410 |
| Nigella sativa | 340 |
| Urtica dioica | 320 |
| Capsella bursa-pastoris | 160 |
| Polygonum bistorta | 190 |

TABLE 3
Lipolysis promoting rate of water extract

| Plant material name | (%) |
|---|---|
| Symplocarpus foetidus | 530 |
| Sanguinaria canadensis | 600 |
| Escholzia california | 730 |
| Mahonia Aquifolium | 460 |
| Myristica fragrance | 780 |
| Musa paradiciaca (fruit site) | 600 |

TABLE 4
Lipolysis promoting rate in the case of combining two
kinds or more of 50% ethanol extracts

| Plant material name | (%) |
|---|---|
| Myristica fragrance + Musa paradiciaca (1:1) | 1900 |
| Myristica fragrance + Sanguinaria canadensis (1:1) | 2200 |
| Musa paradiciaca + Escholzia california (1:1) | 1200 |
| Sanguinaria canadensis + Escholzia california (1:1) | 2100 |
| Musa paradiciaca + Sanguinaria canadensis (1:1) | 1700 |
| Myristica fragrance + Escholzia california + Sanguinaria | 2400 |
| canadensis (1:1:1) | |
| Escholzia california + Sanguinaria canadensis + Musa | 1500 |
| paradiciaca (1:1:1) | |
| Sanguinaria canadensis + Myristica fragrance + Musa | 1800 |
| paradiciaca (1:1:1) | |
| Myristica fragrance + Musa paradiciaca + Escholzia | 2200 |
| california + Sanguinaria canadensis (1:1:1:1) | |

Hereinafter, while the present invention will be described in more detail with examples, it is to be understood that the scope of the present invention is not intended to be limited by these examples.

### EXAMPLE 1

Chewing gum was prepared according to the following formula.

| | | |
|---|---|---|
| Gum base | 20.0 | parts |
| Sugar | 55.0 | parts |
| Glucose | 23.7 | parts |
| Softner | 1.0 | part |
| Mahonia Aquifolium 50% ethanol extract | 0.8 | parts |

### EXAMPLE 2

Chewing gum was prepared according to the following formula.

| | | |
|---|---|---|
| Gum base | 20.0 | parts |
| Xylitol | 75.0 | parts |
| Reduced maltose | 3.8 | parts |
| Softner | 1.0 | part |
| Mahonia Aquifolium water extract | 0.2 | parts |

### EXAMPLE 3

Tablet confectionery was prepared according to the following formula.

| | | |
|---|---|---|
| Sugar | 75.0 | parts |
| Lactose | 20.0 | parts |
| Glycerine fatty acid ester | 0.2 | parts |
| Flavor | 0.4 | parts |
| Nasturtium officinale 50% ethanol extract | 0.1 | parts |
| Purified water | 4.3 | parts |

### EXAMPLE 4

Chocolate was prepared according to the following formula.

| | | |
|---|---|---|
| Sugar | 41.0 | parts |
| Chocolate liquor | 15.0 | parts |
| Whole milk powder | 25.0 | parts |
| Cocoa butter | 18.0 | parts |
| Emulsifier | 0.3 | parts |
| Flavor | 0.4 | parts |
| Symplocarpus foetidus 50% ethanol extract | 0.3 | parts |

### EXAMPLE 5

A drink was prepared according to the following formula.

| | |
|---|---|
| Fructose-glucose drink | 5.00 parts |
| Sugar | 4.50 parts |
| Acidulant | 1.28 parts |
| Flavor | 0.20 parts |
| Polygonum bistorta 50% ethanol extract | 0.02 parts |
| Purified water | 89.0 parts |

### EXAMPLE 6

A drink was prepared according to the following formula.

| | | |
|---|---|---|
| Orange juice | 85.25 | parts |
| Sugar | 11.70 | parts |
| Citric acid | 2.00 | parts |
| Flavor | 1.00 | part |
| Myristica fragrance 50% ethanol extract | 0.05 | parts |

### EXAMPLE 7

An ice cream was prepared according to the following formula.

| | |
|---|---|
| Fructose-glucose liquid sugar | 0.5 parts |
| Sugar | 8.7 parts |
| Acidulant | 1.2 parts |
| Flavor | 0.3 parts |
| Purified water | 89.0 parts |
| Stabilizer | 0.2 parts |
| Nigella sativa water extract | 0.1 parts |

### EXAMPLE 8

Dog food was, prepared according to the following formula.

| | |
|---|---|
| Corn | 33.0 parts |
| Flour | 35.0 parts |
| Soybean meal | 21.0 parts |
| Rice bran (defatted) | 5.5 parts |
| Meat meal | 5.0 parts |
| Mineral mix | 0.2 parts |
| Unonopsis floribunda 50% ethanol extract | 0.3 parts |

### EXAMPLE 9

A capsule was prepared according to the following formula.

| | |
|---|---|
| Symplocarpus foetidus water extract | 50.0 parts |
| Lactose | 48.0 parts |
| Magnesium stearate | 2.0 parts |

The above ingredients were uniformly mixed, and the mixed powder thereof was filled into a hard capsule.

### EXAMPLE 10

A tablet was prepared according to the following formula.

| | |
|---|---|
| Urtica dioica 50% ethanol extract | 20.0 parts |
| Fine grain for direct tableting | 48.0 parts |

(magnesium aluminometasilicate 20%, corm starch 30%, and lactose 50%)

| | |
|---|---|
| Crystalline cellulose | 30.0 parts |
| Magnesium stearate | 2.0 parts |

The above ingredients were uniformly mixed, and the mixed powder thereof was tableted into a tablet of 200 mg/tablet.

### EXAMPLE 11

A syrup was prepared according to the following formula.

| | |
|---|---|
| Myristica fragrance water extract | 0.1 parts |
| Simple syrup | 30.0 parts |
| Purified water | 69.9 parts |

The above plant extract was completely dissolved in the purified water, and then the simple syrup was added and mixed to obtain the corresponding syrup.

### EXAMPLE 12

A candy was prepared according to the following formula.

| | |
|---|---|
| Escholzia california water extract | 0.2 parts |
| Sugar | 50.0 parts |
| Glutinous starch syrup | 35.3 parts |
| Flavor | 0.5 parts |
| Purified water | 14.0 parts |

### EXAMPLE 13

A biscuit was prepared according to the following formula.

| | |
|---|---|
| Myristica fragrance 50% ethanol extract | 0.5 parts |
| Flour | 50.6 parts |
| Corn Starch | 5.1 parts |
| Sugar | 12.7 parts |
| Margarine | 6.5 parts |
| Salt | 0.3 parts |
| Sodium carbonate | 1.3 parts |
| Ammonium carbonate | 0.5 parts |
| Soybean lecithin | 0.3 parts |
| Whole egg | 4.1 parts |
| Flavor | 0.3 parts |
| Purified water | 17.8 parts |

The above materials were mixed to form dough, and spread, followed by being molded and roasted in an oven to produce the corresponding biscuit.

1-3. (canceled)

4. A lipolysis promoter containing Symplocarpus foetidus as active ingredient.

5. A lipolysis promoter containing Symplocarpus foetidus, and one kind, or two or more kinds of plant bodies selected from the group consisting of Myristica fragrance, Escholzia california, Sparganium stoloniferum, Sanguinaria canadensis, Mahonia Aquifolium, Acorus gramineus, the fruit site of Musa paradiciaca, Cytisus scoparius, Hydrastis canadensis, Ficaria ranunculoides, Fumaria officinalis, Unonopsis floribunda, Nasturtium officinale, Nigella sativa, Urtica dioica, Capsella bursa-pastoris, and Polygonum bistorta as active ingredients.

6. A lipolysis promoter containing one kind, or two or more kinds of plant bodies selected from the group consisting of Myristica fragrance, Escholzia california, Sparganium stoloniferum, Sanguinaria canadensis, Mahonia Aquifolium, Acorus gramineus, the fruit site of Musa paradiciaca, Cytisus scoparius, Hydrastis canadensis, Ficaria ranunculoides, Fumaria officinalis, Unonopsis floribunda, Nasturtium officinale, Nigella sativa, Urtica dioica, Capsella bursa-pastoris, and Polygonum bistorta as active ingredients.

7. A lipolysis promoter containing water and/or organic solvents extracts of the plant body of Symplocarpus foetidus according to claim 4 as active ingredient.

8. A lipolysis promoter containing water and/or organic solvents extracts of the plant bodies according to claim 5 as active ingredient.

9. A lipolysis promoter containing water and/or organic solvents extracts of the plant bodies according to claim 6 as active ingredient.

10. Food and drink containing the lipolysis promoter according to claim 4.

11. Food and drink containing the lipolysis promoter according to claim 5.

12. Food and drink containing the lipolysis promoter according to claim 6.

13. Food and drink containing the lipolysis promoter according to claim 7.

14. Food and drink containing the lipolysis promoter according to claim 8.

15. Food and drink containing the lipolysis promoter according to claim 9.
